(19) United States
(12) Patent Application Publication
(10) Pub. No.: US 2013/0197468 A1
(43) Pub. Date: Aug. 1, 2013

(54) Device for Intramyocardial Delivery

(71) Applicants: David SCHWARTZMAN, Pittsburgh, PA (US); Lee E. Weiss, Pittsburgh, PA (US); Phil Gordon Campbell, Cranberry Township, PA (US); Gary K. Fedder, Turtle Creek, PA (US)

(72) Inventors: David SCHWARTZMAN, Pittsburgh, PA (US); Lee E. Weiss, Pittsburgh, PA (US); Phil Gordon Campbell, Cranberry Township, PA (US); Gary K. Fedder, Turtle Creek, PA (US)

(73) Assignees: CARNEGIE MELLON UNIVERSITY, Pittsburgh, PA (US); University of Pittsburgh-of the Commonwealth System of Higher Education, Pittsburgh, PA (US)

(21) Appl. No.: 13/751,414

(22) Filed: Jan. 28, 2013

Related U.S. Application Data
(60) Provisional application No. 61/590,938, filed on Jan. 26, 2012.

(51) Int. Cl. A61M 5/00

(52) U.S. Cl. CPC A61M 5/00
USPC 604/500; 424/93.72; 514/773; 514/772; 424/93.1; 424/130.1; 604/93.01

(57) ABSTRACT

Methods and apparatuses for delivery of biologically active material and/or sensors to a target organ or system. The apparatuses allow for specific, controlled delivery of the biologically active material and targeted placement of sensors. The apparatuses may be fabricated from cellular and/or acellular biological active components to promote integration of sensors into tissue and achieve appropriate release of biologically active molecules. The apparatuses may be fabricated from plasma-containing materials or other biopolymers such that the apparatus will resorbed into the tissue following insertion. The biologically active cellular or acellular component may be incorporated into that material may then serve as the source of the therapeutic biologically active component. The apparatus may take the form of a screw, though numerous shapes arc contemplated. The delivery methods and apparatuses of the present invention may be employed in a wide variety of physiological and medical situations, with cardiac implementations being particularly appropriate.

### CROSS-REFERENCE TO RELATED APPLICATIONS

This application claims priority to U.S. Patent Application Ser. No. 61/590,938 filed on Jan. 26, 2012.

### GOVERNMENT SUPPORT

This invention was made with government support under grant number 2 UL1 RR024153-06 awarded by the National Institutes of Health. The government has certain rights in the invention.

### BACKGROUND OF THE INVENTION

1. Field of the Invention

The present invention relates generally to the field of cardiac health and specifically to the field of heart monitoring and/or improving heart function and/or structure.

2. Description of the Background

Cardiovascular disease (CVD) is the leading cause of death in the western world. In the U.S. alone, patients suffer nearly 800,000 heart attacks per year and more then 2,200 Americans die of CVD each day. New therapeutic strategies for cardiac repair and monitoring are clearly needed.

Contemporary therapies include delivery of cellular and acellular biomaterials. Rejuvenative therapy by injection of cells has shown great promise, however, without delivering cells in a matrix (scaffold), cell retention, survival, and integration into the host tissue may be problematic. Further research has established that delivery of cells in a matrix can improve cellular retention and viability. While large-scale regeneration will likely require an exogenous cell source, many studies have also shown improvements with a matrix alone. There are numerous biomaterial matrices that have been investigated for cell delivery or used alone, including native, synthetic, and biomimetic materials. See Rane and Christman, “Biomaterials for the treatment of myocardial infarction: a 5-year update.” J. Am. Coll. Cardiol., 2011. 58(25):2615-29 for comprehensive survey, which is hereby incorporated by reference. The advantages of using native biomaterials are their constituents provide a microenvironment that contains physical, chemical, and mechanical cues similar to native tissue.

While both transepicardial and transendocardial deployments of matrices are feasible, epicardial delivery is highly invasive, with associated risks and high costs. Minimally invasive transendocardial delivery via catheter deployment is a more practical clinical approach. For such applications, the art generally believes that the material should be an injectable hydrogel precursor that has the proper gelation properties and kinetics to remain liquid within the catheter during delivery, while allowing the formation of a gel within the myocardial tissue following delivery. Several problematic issues arise with this approach: 1) gel injectability, i.e., assuring gelation does not prematurely occur in the catheter or that the material does not leak out of the catheter prior to injection; 2) maintaining gel localization at the delivery site, i.e., assure that the gel precursor remains in targeted injection locations and does not egress through the path formed by needle puncture: 3) gel retention for sufficient time needed for regeneration, i.e., does not degrade too quickly; and, 4) inherent gel stiffness properties, which may not be optimal for physiological circumstances.

One injectable considered for clinical use is based on blood plasma platelet gel. Cheng, et al., “Intramyocardial injection of platelet gel promotes endogenous repair and augments cardiac function in rats with myocardial infarction.” J. Am. Coll. Cardiol., 2012, 59(3): 256-64. This approach is similar in spirit to autologous platelet-rich plasma (aPRP) therapies currently used in sports medicine and wound healing applications. Those aPRP therapies are controversial because of the outcomes have been highly variable and unpredictable, due in past to patient-to-patient variability in platelet quantity and quality, bed-side preparation variability, and their relatively short residence times in vivo. Additionally, administration of aPRP-based formulations is traditionally more difficult as such products are not an off-the-shelf solution. Blood plasma platelet gels used in cardiac repair may suffer similar consequences, and, when used in these cardiac applications, the blood plasma/platelets must be pre-activated just prior to injection; therefore gelation kinetics and injection timing may slid be problematic for reliable deployment.

In addition, significant advances have been made in the development of materials and devices which are capable of improving tissue structure, healing, and function, as well as monitoring or protecting the tissue. Examples include heart monitoring devices that are able to measure cardiac motion, pressure, or wall stress. Placement of sensors in the heart has previously been limited to the right side of the organ due to concerns of increasing the risk of thrombosis, embolization, and sensor stability.

The present invention addresses the deficiencies of the prior art by providing novel methods and apparatuses by which materials may be administered to the heart, other solid organ, or tissue of a patient in need thereof. The present invention employs apparatuses that may controlably deliver biologically active materials or sensors directly to specific locations in the organ or tissue, e.g. heart or muscle. The apparatuses may be fabricated from the cellular and acellular biologically active components to promote appropriate integration into the tissue, healing of the tissue, and appropriate temporal and spatial administration of biologically active molecules. The delivery methods and apparatuses of the present invention may be employed in a wide variety of physiological and medical situations.

### SUMMARY OF THE INVENTION

The present invention addresses the deficiencies of the prior art by providing novel methods and apparatuses by which materials may be administered to the heart, other solid organ, or tissue of a patient in need thereof. The present invention employs apparatuses that may controllably deliver biologically active materials or sensors directly to specific, locations in the organ or tissue, e.g. heart or muscle. The apparatuses may be fabricated from the cellular and acellular biologically active components to promote appropriate integration into the tissue, healing of the tissue, and appropriate temporal and spatial administration of biologically active molecules. The delivery methods and apparatuses of the present invention may be employed in a wide variety of physiological and medical situations, with cardiac implementations being particularly appropriate.

The apparatus may be fabricated from plasma-containing materials or other biopolymers such that the apparatus will be resorbed into the tissue following insertion. In some embodiments, the biologically active cellular or acellular component is incorporated into the material from which the apparatus is fabricated and that material serves as the source of the biologically active component. The apparatus may be in form of nails, rings, loops, screws, barbs, or any shape that is appropriate for the particular application confronting the medical practitioner. In some embodiments, the apparatus may include a hollow chamber inside. The apparatus may be implanted directly into a target organ or tissue through the vasculature. The hollow chamber within the apparatus may contain the biologically active materials that are to be delivered to the patient, instead of or in addition to the biologically active materials contained in the apparatus body. In certain contexts, it may be desirable for the apparatuses to include two components—a harder tip having physical properties conducive to insertion into more resilient or hard tissues (e.g., heart wall, bone), while preserving a different set of properties for the body of the apparatus that may achieve the desired pharmacokinetic release properties appropriate for the medical context confronting the medical practitioner.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 shows a hidden-line view of an apparatus of the present invention;

FIG. 2 displays a hidden-line view of a second embodiment of the present invention having multiple chambers;

FIG. 3 depicts an embodiment of the present invention having a tip with harder physical properties than the main invention;

FIG. 4 shows an embodiment of the present invention having a physiologic sensor; and

FIG. 5 shows the placement of embodiments of the present invention in the wall of a human heart.

### DETAILED DESCRIPTION OF THE INVENTION

It is to be understood that the figures and descriptions of the present invention have been simplified to illustrate elements that are relevant for a clear understanding of the invention, while eliminating for purposes of clarity, other elements that may be well known. The detailed description will be provided hereinhelow with reference to the attached drawing.

The present invention provides methods and apparatuses for the improved treatment of solid internal organs, including the heart. The present invention further provides methods and apparatuses for the improved treatment of tissues into which the devices of the present invention may be secured. The present invention is useful tor the precise placement of sensors in the organ or tissue tor extended monitoring. The present invention is also useful in the delivery of native and exogenous biologically active materials to the solid organ or tissue both reliably and precisely. The apparatus may be fabricated from or a biologically active material. The body of the apparatus may also be fabricated from cell-based formulations or contain as a major or minor component a biologically active material, as described below. In those circumstances, the apparatus itself is the both delivery vehicle and source of biologically active material.

While the description below emphasizes the implementation of embodiments for the heart, the present invention may be employed in the monitoring or treatment of any solid organ, (e.g., heart) or tissue (e.g., muscle, brain). Additionally, the term “patients” as used herein is meant to encompass not only humans, hut any animal where the monitoring or treatment of a solid organ or tissue is desired.

The present invention encompasses devices that may be implanted into a solid organ or tissue tor the targeted delivery of biologically active components to that target organ or tissue or the targeted placement of a physiologic sensor into that location. The devices of the present invention may deliver cellular or acellular biologically active components through the device or alternatively, the body of the device may be fabricated from a biologic material itself. Those acellular biologically active components may generally referred to as a “drug” and include, for example, a wide variety of synthetic and natural components, including small molecules, peptides and proteins (including antibodies and extracellular matrix proteins), and nucleotides. In some embodiments, the apparatuses of the present invention may include one or more internal chambers in which the biologically active component is housed during placement of the apparatus. As the body of the apparatus degrades, the cellular or acellular material in the chamber(s) may escape and be delivered to the patient at the location in the body where the device has been implanted. Thus, the hollow chambers may act as reservoirs tor biologically active materials to be delivered to the patient. Additionally, the devices may include a composition that facilitates the integration of the device into the wall of the organ or tissue and reduces rejection of the device by the body.

The apparatuses of the present invention may be secured into the wall of a target organ or into any tissue. In specific implementations, the devices of the present invention may be implanted into heart tissue via either endocardial or epicardial methods. For other placement of the devices of the present invention into non-cardiac locations, the devices of the present invention may similarly be deployed either transvascularly or directly into the organ or tissue. In some embodiments, the devices of the present invention may be employed via a catheter, as discussed further below.

The devices of the present invention may be fabricated from a variety of materials. The material may be biodegradable material. In some embodiments, the devices of the present invention may be fabricated from both metal and a biodegradable substance. The metal may provide structural stability to the target organ or tissue, such as in implementations when the devices of the present invention are placed into bone. Similarly, the physical properties of the material (e.g., stiffness and porosity) may be manipulated to match the appropriate circumstance confronting the medical practitioner.

In certain embodiments, the apparatuses of the present invention are fabricated from a biodegradable material that is slowly resorbed into the body over time. The material from which the devices are fabricated may he designed to attenuate any unwanted reaction of the patient's body (e.g., inflammation) as to permit better integration of a device (e.g., pressure sensor) or biologic (e.g., cells) into the target organ or tissue.

The devices of the present invention may be used to deliver biologically active components to a target organ or tissue in other manners as well. As noted above, the chamber or chambers of the devices of the present invention may include a single or numerous biologically active materials. Additionally, the shell of the device may be fabricated from a therapeutic biologic material. For example, its some embodiments, the shell of the device may be fabricated from a plasma-derived composition, as disclosed in U.S. Pat. No. 8,293,530, which is hereby incorporated by reference.

Plasma-based materials (PBM) may be manufactured starting with pooled, allogenic blood plasma and platelet fraction. Unlike platelet concentrators used in aPRP therapies, which concentrate the platelets and subsequently discard a significant volume of plasma, PBMs employed within the context of the present invention may utilize the entire plasma, including the platelets, which will often include growth factors and other components that are not platelet-derived. Gelling with calcium chloride to saturate the sodium citrate anticoagulate activates the platelets, and the resulting hydrogel is freeze-dried and ground into a powder. The powdered material is then mixed with glycerol as a plasticizer. The resultant mixture forms a dough-like material that is compression molded at low temperatures (approximately 50-70° C.) and high-pressures (approximately 2,000 to 10,000 psi) into highly concentrated, dense solid forms as shelf-stable PBM that retain native biological activity.

Prior to molding, additional components may be added to the dough, as needed, including porogens, cross-linking agents, other ECM powders (e.g., cardiac ECM; “CECM”), minerals, growth factors, or immunoresponse regulators such as IL-10. For example, the elution rate of drugs from PBMs, PBM mechanical properties, and degradation rates can be further modulated with the addition of the plant-based cross-linker genipin (see, e.g., U.S. Pat. No. 8,293,530) which is substantially less toxic than glutaraldyde used in the prior art to control CECM gel stiffness. Porous bioplastics may be created by adding mannitol powder to the mix; after hydration, the mannitol dissolves in vitro or in vivo. For cell delivery, cells may be seeded on porous PBMs after the molding process. To assure safety, PBMs may also virally inactivated using a combination of pasteurization and gamma radiation as an added safety precaution against the remote possibility that any of the donated and screened blood products arc contaminated with unknown pathogens. Using pooled plasma/platelets overcomes patient-to-patient plasma/platelet variability associated with autologous sourcing approaches. In addition, use of pooled PRP enables these products to be rapidly available for off-the-shelf use and lowers manufacturing costs, which translates into relatively low cost products.

This composition may be useful in treating a medical condition in the solid organ or tissue in which the device is placed and/or promote incorporation of the apparatus into the local biological environment. When fabricated from a PBM, the device of the present invention may include a coating that maintains the integrity of the device daring any medical procedures employed to insert the device into the target organ or tissue. The devices of the present invention may be fabricated in whole or in part of a PBM or other compositions that include platelets or other biologically derived materials as a minor component.

The apparatuses of the present invention may also be fabricated from bioplastics processing technology that can form native protein and peptide (i.e., proteinaceous) materials into complex three-dimensional shapes using a low-temperature, high-pressure compression molding process that maintains bio-activity of proteins. Such bioplastic materials become rubbery upon hydration, however, their microstructural, mechanical, and degradation properties can be modified by varying the relative amounts of plasticizer, porogens, or plant-based cross-linkers mixed in with the proteinaceous material prior to molding of the device. That manufacturing process also enables growth factors, immunoresponse regulators, and drugs to be directly composited with bioplastics. The bioplastic may include ECM or CECM as a major or minor component. Additionally, porous bioplastics could be seeded with cells, post-molding, to enhance cell delivery.

The materials that make up the device may be in part or in whole a therapeutic material that provides a benefit to the organ or tissue into which it is placed, e.g., plasma-based materials, formulations containing ECM or CECM. The devices of the present invention may also be fabricated from a material that is able to achieve a controlled release of a biologically active agent into the target organ or tissue. Apparatuses fabricated from PBMs are believed to be degraded by the patient's body by proteolysis. As such, control over release of a biologically active agent is achieved through cellular-mediated enzymatic digestion of the apparatus. When fabricated from biologically compatible plastics, such as, but not limited to, those PBM compositions described above, the apparatuses of the present invention may be fabricated using a compression molding approach.

The apparatuses of the present invention may be formed into numerous shapes. The apparatuses may be in the form of nails, rings, screws, loops, barbs, or any shape that is appropriate for the particular application confronting the medical practitioner. The specific fixation approach employed may be adapted to satisfy any anatomical requirements of the tissue substrate into which the apparatuses are being delivered. In many medical applications, screws are used to implant a device at a particular location in the patient's body. While some of the description below centers on implementations of the present invention having the structure of a screw, the general concepts are equally applicable to any shape of apparatus that might be needed for the application at hand.

The present invention will provide a clinically practical approach for highly controlled, localized delivery of biologically active materials because the placement of these devices will not rely on gelation kinetics of a formulation. Instead, the apparatus will preferably ‘lock’ into place of deployment of the biologically active material at the site of implanting. The inherent bioactivity of the apparatuses of the present invention fabricated from plasma- or protein-based materials may be sufficient to aid repair or could be used to enhance the actions of other biologic components such as cells and growth factors dub these screws could deliver. Moreover, bioplastic apparatuses of the present invention have tailorable degradation and mechanical properties, and represent off-the-shelf devices, storable at room temperature, that are easily and conveniently employed by medical practitioners.

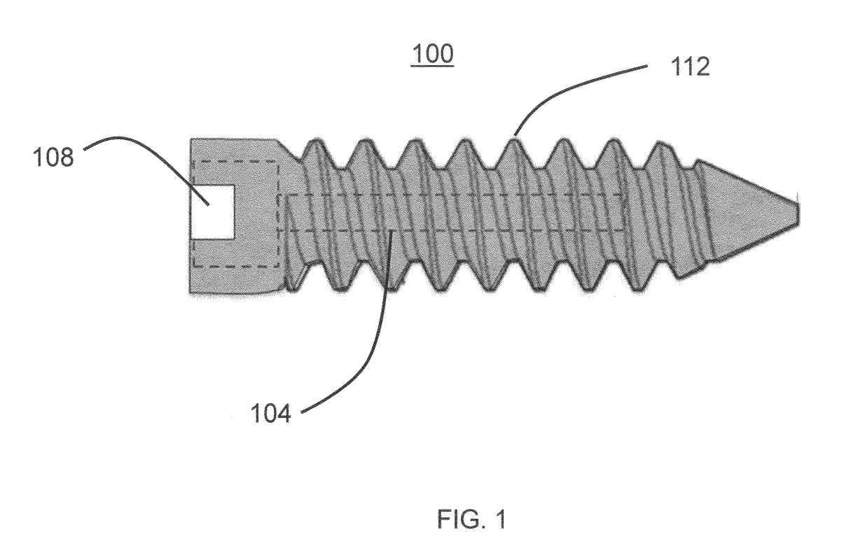

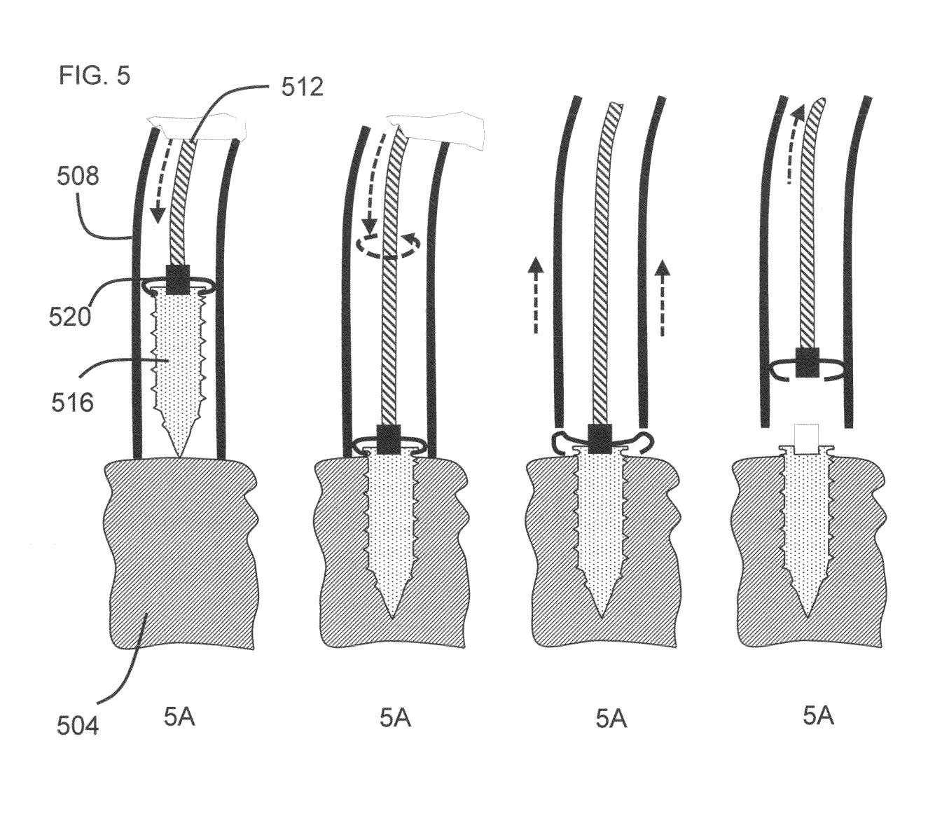

A screw-shaped embodiment 100 of the present invention is shown in FIG. 1. The outside of the screw may contain threads 112 to allow implantation of the apparatus into the target tissue or organ. The apparatus 100 includes an internal chamber 104 that is hollow. That chamber 104 may contain a biologically active material for delivery to the organ or tissue. As noted above, the apparatus 100 may also be fabricated from a PBM or a material into which a biologically active agent is incorporated, such that the apparatus body is used to deliver material to the target organ or tissue. In other embodiments, the chamber 104 may include a solution of a chemotherapeutic agent tor localized delivery to a cancerous organ, such as the pancreas. In some embodiments, the head of the screw will contain a square slot 108 feature that may be mated with a driver bit attached to a guide wire, as described below with regards to FIG. 5.

As noted above, the present invention is a generalized tool for the delivery of such cellular and acellular materials to solid organs and tissues. As such, the specific components that may be included in the internal chamber of the apparatuses of the present invention may be tailored to the specific physiological or disease state that confronts the treating physician.

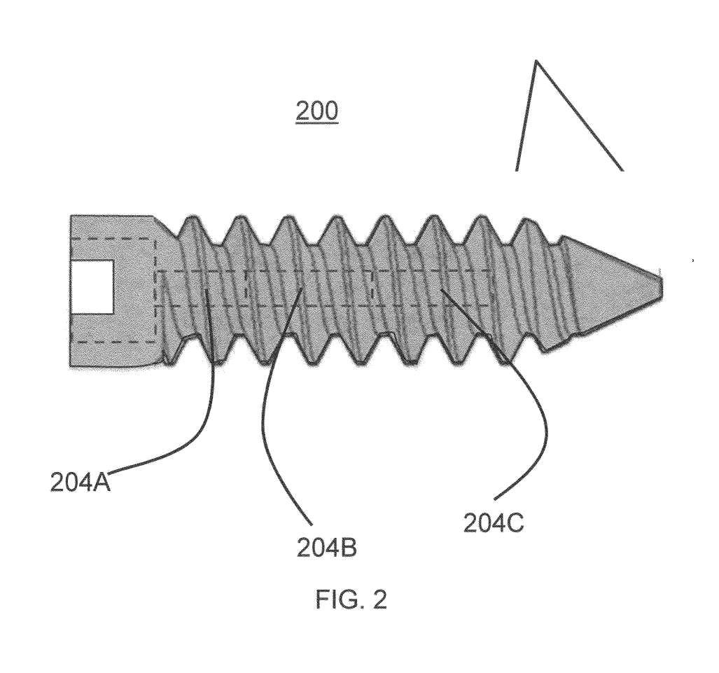

In other embodiments, the device may include one or mom chambers incorporated within the body of the device 200 as shown in FIG. 2. Each chamber 204A, 204B, 204C may include a different biologically active compound component (e.g. cellular or acellular components). Each component may work synergistically with another component to address a single medical condition at that organ or tissue. Alternatively, each component may work independently to treat distinct medical conditions present in the same organ or tissue. The components in each of the chambers 204A, 204B, 204C may be released simultaneously or sequentially as dictated by the treating physician and the condition or conditions being treated.

In some instances, it may be desired to have a particular stiffness and porosity of screw (or other apparatus shape) that might be incompatible with insertion of that screw into the target tissue or organ. Such circumstances may arise when the target tissue or organ (e.g., bone or cardiac wall) dictates stiffer or more resilient properties tor insertion into the tissue or penetration of the organ. At the same time, those stiller physical properties may result in apparatuses have other properties (e.g., dissolution rate, drug release rate, or porosity) that are incompatible with the medical application for which the apparatuses are being employed. To address such situations, the present invention provides apparatuses that may include multiple components—for example, one having the appropriate physiological and physical properties for the medical application and a second having the physical components appropriate for penetrating the target tissue or organ.

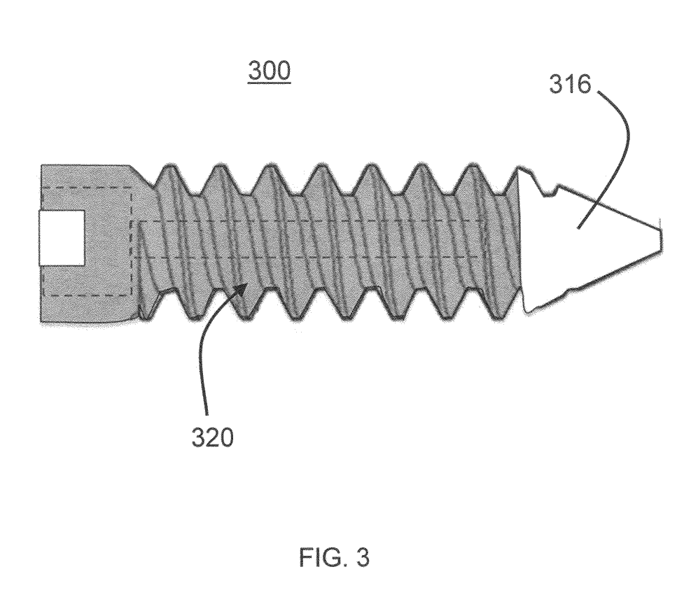

An example of such an embodiment 300 is shown in FIG. 3. The screw-shaped embodiment 300 of the present invention shown in FIG. 3 includes a tip 316. The tip 316 may be fabricated from a harder biocompatible material than the main body of the apparatus 320. For example, the main body 320 may be formulated from PBMs and the screw tip 316 may be formulated from polylactic acid (PLA), polyglycolic acid (PGA), or combinations of both. In some embodiments, a 50/50 mixture of PLA/PGA is used in fabricating the screw tip. The tip is expected to remain sharp in aqueous environments for a sufficient amount of time to permit the apparatus to be deployed at the appropriate location in the target tissue or organ. Over time, however, the tip will lose sharpness through degradation over the course of several weeks after placement in the body. In the apparatus shown in FIG. 3, the tip 316 may be co-molded with the body 320 of the screw such that the two perform as a single apparatus.

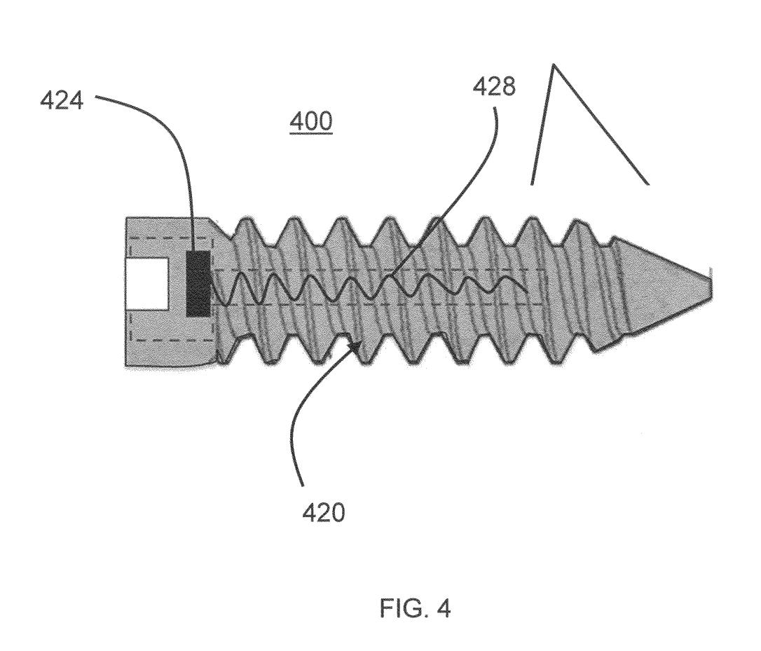

In some embodiments, such as shown in FIG. 4, the apparatuses 400 of the present invention may be used to place a physiological sensor 424 in the solid organ or tissue. When employed in that manner, the outer attachment component 420 (e.g., hollow screw) may be used to initially secure the sensor 424 in the desired location. As the main body of the apparatus 420 degrades over time, the body will heal from the insertion and the sensor 424 may be integrated into the location within the body. This property will facilitate the long-term placement and maintenance of the sensor 424 within the target organ or tissue. When made from metal, the shaft of the implantation device of the present invention may be used as an antenna to transmit information from a sensor 424 placed in the heart.

An example of such an embodiment is shown in FIG. 4. There, the body of the fixation portion 420 of the device may house an internal wire 428. The wire 428 may be in the form of a corkscrew or “pig tail” and may be used as an antenna to communicate physiological data to a receiver located distal from the target location. Additionally, the corkscrew may also help to fix the location of the device as the body of the apparatus degrades over time. In alternative embodiments, the antenna may be a straight wire, a corrugated wire, or any geometry that may be appropriate for the specific implementation desired by the medical practitioner. The transmission circuitry may be adapted to communicate using a wide variety of telecommunication protocols (e.g., RF, BLUETOOTH) and includes the appropriate components to accomplish that transmission. In some embodiments, near-field magnetic induction communication may also be employed. In certain implementations, devices that include a pressure sensor may be placed into a cardiac wall to measure intraventricular pressure or stress. Thus, the present invention may be used for long-term monitoring of cardiac pressure.

Other physiological sensors may be employed in the context, of the present invention, including pH sensors, glucose sensors, temperature sensors, flow sensors, biochemical sensors, motion sensors, and others. Additionally, the device to be implanted into the target organ or tissue may include a mechanism for generating power for use by the sensor and transmission circuitry. The sensor may employ specialized materials and circuitry (e.g., piezoelectric circuitry) to generate power for the sensor to employ. The piezoelectric circuitry may scavenge energy from heart wall contractions or horn heart wall motion, or from blood flow inside the heart. As one embodiment, wire 428 in FIG. 4 may be used as part of a piezoelectric circuit to scavenge energy from the heart wall contractions. Alternatively, a wire may extend into the ventricle cavity and be attached to the body of the fixation portion 420 of the device in order to scavenge energy from blood flow. The specific electronic components that may be used within the context of the present invention may be tailored to the physiological or diseased state that the practicing physician wishes to assess.

As noted above, the devices of the present invention may be deployed either transvascularly or directly into the organ or tissue. In the specific circumstance of cardiac deployment, the screw apparatuses of the present invention may be inserted either transepicardially or transendocardially. An example of transendocaridal implantation is shown in FIG. 5. In some embodiments, a screw may be guided to the endocardium 504 using a clinically available steerable catheter 508. This catheter would be appropriately modified at its proximal end (i.e., end closer to the user and away from the patient, details not shown) to accept screw insertion into the catheter 508 and a motor-driven mechanism to automatically advance and drive a high-torque guide wire 512 to which the screw 516 may be mated.

A spring clasp 520 may be used to keep the screw 516 engaged with the guide wire 512 to prevent the screw 516 from disengaging until it is secured in the myocardium 504, or, if needed, to back it out. The following insertion sequence may be employed: (1) deploy catheter 508 to desired screw delivery site; insert screw/guide-wire 512/516 into catheter 508 and advance the screw 516 until the screw/catheter 516/508 is in contact against endocardium 504—FIG. 5A; (2) advance and rotate the guide-wire 512 until screw 516 is deployed in the myocardium 504—FIG. 5B; (3) pull-back catheter 508 to disengage spring clasp 520 from screw 516—FIG. 5C; and, (4) pull guide-wire 512 back out FIG. 5D. At this point the catheter may be repositioned for deployment the next screw, and the sequence repeated as needed.

Because the present invention may be firmly fixed in the wall of a patient's organ or into tissue, the placement may be stably maintained after implantation. This attribute is of particular benefit when treating or monitoring organs that move significantly, such as the heart.

Because the apparatuses of the present invention are a solid-phase material, they may be fixed into a wall of a solid organ or into tissue. As a result, issues that accompany liquid-phase delivery vehicles, such as migration of the injected solution and subsequent dilution of any delivered biologically active material are mitigated. Accordingly, the present invention is able to achieve reliable and precise administration of biological active agents to a target tissue. Furthermore, the present invention provides for the controlled degradation of biologically active components over time.

Nothing in the above description is meant to limit the present invention to any specific materials, geometry, or orientation of elements. Many modifications are contemplated within the scope of the present invention and will be apparent to those skilled in the art. The embodiments described herein were presented by way of example only and should not be used to limit the scope of the invention.

We claim:

1. An apparatus to be implanted into a target solid organ or tissue, comprising:
an apparatus body;
a mechanism capable of implanting said apparatus to said target solid organ or tissue; and
a source of biologically active material.

2. The apparatus of claim 1, wherein said apparatus body is fabricated from a biodegradable material.

3. The apparatus of claim 1, wherein said biodegradable material is a bioplastic.

4. The apparatus of claim 3, wherein said bioplastic is a plasma-based material.

5. The apparatus of claim 4, wherein said plasma-based material is a platelet-rich plasma-based composition.

6. The apparatus of claim 3, wherein said bioplastic contains a proteinaceous material.

7. The apparatus of claim 3, wherein said bioplastic includes extracellular matrix components.

8. The apparatus of claim 7, wherein said extracellular matrix components are cardiac extracellular matrix components.

9. The apparatus of 2, wherein said biodegradable material comprises a cellular component.

10. The apparatus of claim 9, wherein said cellular component comprises therapeutic stem cells or a cellular suspension.

11. The apparatus of claim 2, wherein said biodegradable material comprises acellular components.

12. The apparatus of claim 11, wherein said acellular components comprise biologically active compounds.

13. The apparatus of claim 12, wherein said acellular components promote integration of said apparatus into said target organ or tissue.

14. The apparatus of claim 13, wherein said acellular components include a chemotherapeutic compound.

15. The apparatus of claim 1, wherein said apparatus body is selected from the group consisting of a screw, a nail a ring, a loop, or a barb.

16. The apparatus of claim 15, wherein said apparatus body is a screw and said mechanism are threads on said screw.

17. The apparatus of claim 16, wherein said screw includes a chamber in said screw that is said source of biologically active material.

18. A method of implanting an apparatus into a target organ or tissue of a patient, comprising the steps of:
fabricating a screw from a biodegradable material, wherein said screw includes a source of biologically active material;
engaging said screw with a spring clasp, wherein said spring clasp is attached to an end of a guide wire;
deploying a catheter to a desired screw delivery site in said target organ or tissue;
placing said screw, spring clasp, and guide wire into a catheter;
advancing said screw, spring clasp, and guide wire until said screw is in contact with said target organ or tissue;
advancing and rotating said guide wire until said screw is deployed into said target organ or tissue;
retracting said catheter from said target organ or tissue;
disengaging said spring clasp from said screw; and
retracting said guide wire and said spring clasp.

19. The method of claim 18, wherein said target organ or tissue is the heart.

20. The method of claim 18, wherein said biodegradable material is a bioplastic.

21. The method of claim 20, wherein said bioplastic is a plasma-derived material.

22. The method of claim 21, wherein said plasma-derived material includes platelet-rich plasma.

23. The method of claim 19, wherein said deploying of said catheter step is achieved transepicardially or transendocardially.
